(19) United States
(12) Patent Application Publication
(10) Pub. No.: US 2010/0168582 A1
(43) Pub. Date: Jul. 1, 2010

(54) HIGH FREQUENCY TRANSDUCERS AND METHODS OF MAKING THE TRANSDUCERS

(75) Inventors: Jian R. Yuan, Pleasanton, CA (US); Nickola Goodson, Fremont, CA (US)

Correspondence Address: Boston Scientific Corporation;c/o Frommer Lawrence & Haug LLP, 745 Fifth Avenue, New York, NY, 10151, US

(73) Assignee: Boston Scientific Scimed, Inc., Maple Grove, MN (US)

(21) Appl. No.: 12/345,282

(22) Filed: Dec. 29, 2008

(51) Int. Cl. A61B 8/14; H04R 31/00

(52) U.S. Cl. 600/459; 29/594

(57) ABSTRACT

A method of making an ultrasound transducer includes providing a piezoelectric crystal of PIN-PMN-PT (lead indium niobate-lead magnesium niobate-lead titanate) and etching kerfs into the piezoelectric crystal using a laser. In at least some embodiments, each kerf has a width of no more than 4 μm. The kerfs are filled with a non-piezoelectric material to form an array of piezoelectric elements.

### FIELD

The present invention is directed to the area of transducers for ultrasound imaging systems, devices and systems containing the transducers, and methods of making and using the transducers. The present invention is also directed to PIN-PMN-PT transducers formed using laser etching techniques, as well as devices and systems containing the transducers, and methods of making and using the transducers.

### BACKGROUND

In medical ultrasound applications, such as dermatology, ophthalmology, laparoscopy, intracardiac ultrasound, and intravascular ultrasound, high frequency ultrasound transducers or transducer arrays with broad bandwidth and high sensitivity are often desired for high resolution imaging. In recent years, a variety of advanced technologies have been studied for developing high frequency ultrasound transducers and arrays with high performance. 1-3 piezoelectric composite structures are often the best selection for these transducers as they typically provide higher electric-mechanical coupling coefficients than the bulk material; lower acoustic impedance than the bulk material, and they permit adjustment of one or both of the acoustic and electric impedance of the composite material for a particular application or system. Additionally, 1-3 piezoelectric composite structures can often be easily tailored to form the transducer or array into a desired shape.

Because of these features, methods of making 1-3 piezoelectric composite transducers have been investigated. To develop 1-3 piezoelectric composite structures with a relatively high working frequency and with high acoustic performance, key technical features include a relatively high coupling constant k₃₃ and lateral vibration modes generated by the kerf structure of the 1-3 piezoelectric composite transducers that are at least an octave range away from the operating frequency.

### BRIEF SUMMARY

One embodiment is a method of making an ultrasound transducer that includes providing a piezoelectric crystal of PIN-PMN-PT (lead indium niobate-lead magnesium niobate-lead titanate) and etching kerfs into the piezoelectric crystal using a laser. In at least some embodiments, each kerf has a width of no more than 4 μm. The kerfs are filled with a non-piezoelectric material to form an array of piezoelectric elements.

Another embodiment is an ultrasound transducer that includes a piezocomposite structure and at least first and second electrodes disposed on the piezocomposite structure. The piezocomposite structure includes a plurality of transducer elements separated by kerfs. The transducer elements are PIN-PMN-PT (lead indium niobate-lead magnesium niobate-lead titanate). In at least some embodiments, each kerf has a width of no more than 4 μm. The kerfs are typically filled with a non-piezoelectric material.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of an elongated member of the catheter shown in FIG. 2 with an imaging core disposed in a lumen in the distal end of the elongated member, according to the invention;

FIG. 4 is a schematic perspective view of one embodiment of a transducer with a 1-3 composite structure, according to the invention;

FIG. 5 is a schematic perspective view of one embodiment of a transducer with a 2-2 composite structure, according to the invention; and

FIGS. 6A-6E are schematic cross-sectional views of one embodiment of a method of making a transducer, according to the invention.

### DETAILED DESCRIPTION

The present invention is directed to the area of transducers for ultrasound imaging systems, devices and systems containing the transducers, and methods of making and using the transducers. The present invention is also directed to PIN-PMN-PT transducers formed using laser etching techniques, as well as devices and systems containing the transducers, and methods of making and using the transducers.

Suitable intravascular ultrasound (“IVUS”) and intracardiac echocardiography (ICE) systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Nos. 2006/0100522; 2006/0106320; 2006/0173350; 2006/0253028; 2007/0016054; and 2007/0038111; all of which are incorporated herein by reference.

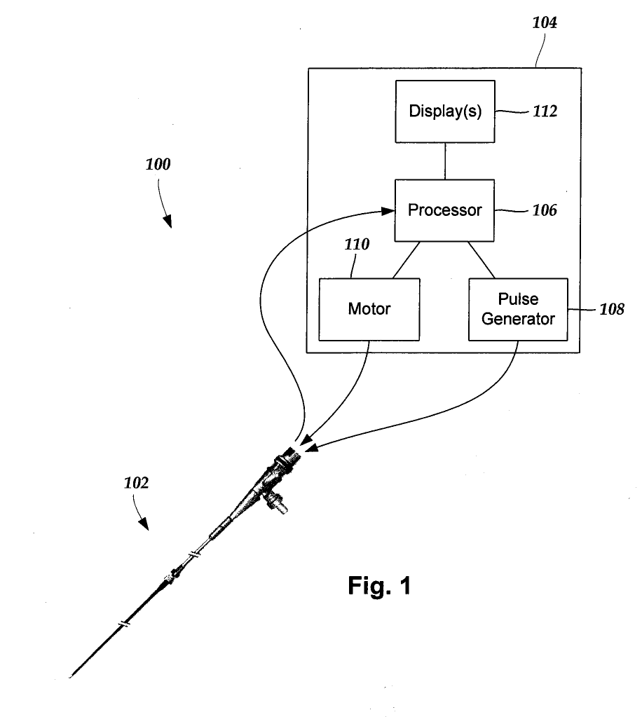

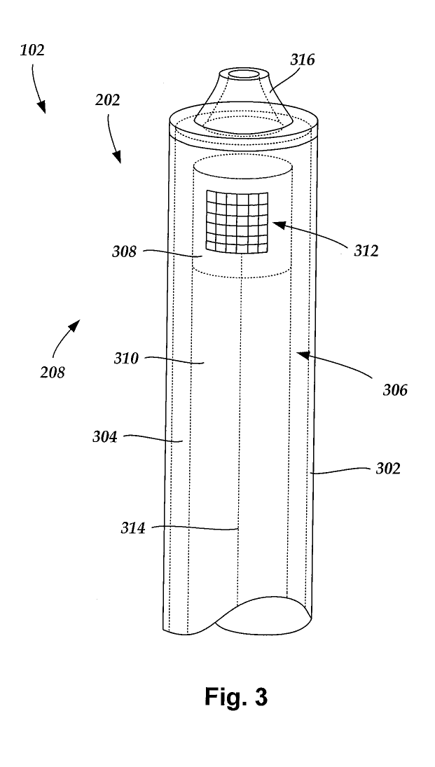

FIG. 1 illustrates schematically one embodiment of an IVUS imaging system 100. An ICE imaging system is similar. The IVUS imaging system 100 includes a catheter 102 that is coupleable to a control module 104. The control module 104 may include, for example, a processor 106, a pulse generator 108, a motor 110, and one or more displays 112. In at least some embodiments, the pulse generator 108 forms electric pulses that may be input to one or more transducers (312 in FIG. 3) disposed in the catheter 102. In at least some embodiments, mechanical energy from the motor 110 may be used to drive an imaging core (306 in FIG. 3) disposed in the catheter 102. In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the motor 110, the velocity or length of the pullback of the imaging core (306 in FIG. 3) by the motor 110, or one or more properties of one or more images formed on the one or more displays 112. In some embodiments, the parts of the control module 104 (i.e., the processor 106, the pulse generator 108, the motor 110, and the one or more displays 112) may be in one unit. In other embodiments, the parts of the control module 104 are in two or more units.

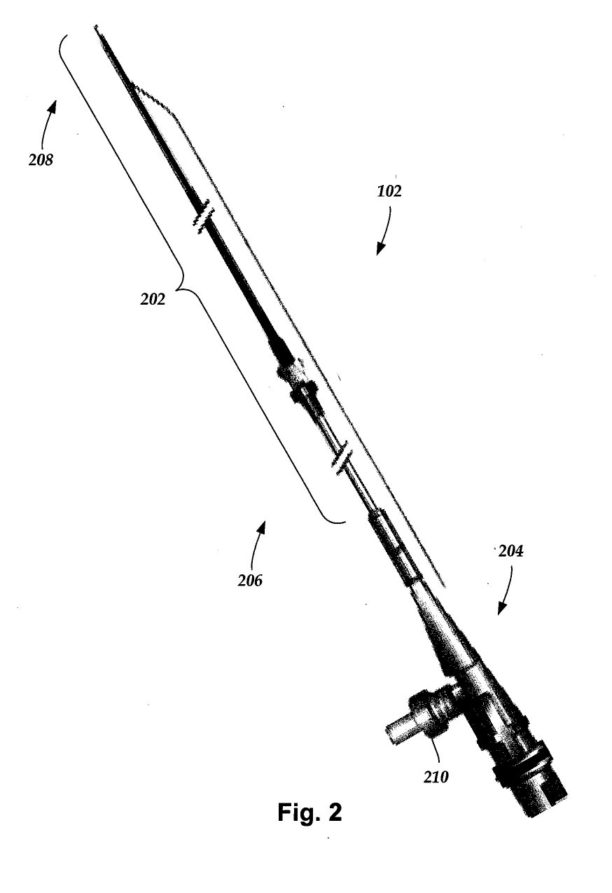

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, polytetrafluoroethylene (PTFE), other plastics, and the like or combinations thereof.

One or more transducers 312 may be mounted to the imaging device 308 and employed to transmit and receive acoustic pulses. In at least one embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

Pressure distortions on the surface of the one or more transducers 312 can be generated in order to form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency or frequencies in one or more selected directions. For example, transducers may be disc-shaped, block-shaped, ring-shaped, and the like.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received. In at least some embodiments, the rotation of the imaging core 306 is driven by the motor 110 disposed in the control module (104 in FIG. 1).

As the one or more transducers 312 rotate about the longitudinal axis of the catheter 102 emitting acoustic pulses, a plurality of images are formed that collectively generate a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may also move longitudinally along the blood vessel within which the catheter 102 is inserted so that a plurality of cross-sectional images may be formed along a longitudinal length of the blood vessel. In at least some embodiments, during an imaging procedure the one or more transducers 312 may be retracted (i.e., pulled back) along the longitudinal length of the catheter 102. In at least some embodiments, the motor 110 drives the pullback of the imaging core 306 within the catheter 102. In at least some embodiments, the motor 110 pullback distance of the imaging core is at least 5 cm. In at least some embodiments, the motor 110 pullback distance of the imaging core is at least 10 cm. In at least some embodiments, the motor 110 pullback distance of the imaging core is at least 15 cm. In at least some embodiments, the motor 110 pullback distance of the imaging core is at least 20 cm. In at least some embodiments, the motor 110 pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 20 MHz to 60 MHz.

In at least some embodiments, one or more conductors 314 electrically couple the transducers 312 to the control module 104 (See FIG. 1). In at least some embodiments, the one or more conductors 314 extend along a longitudinal length of the rotatable driveshaft 310.

In at least some embodiments, the catheter 102 with one or more transducers 312 mounted to the distal end 208 of the imaging core 308 may be inserted percutaneously into a patient via an accessible blood vessel, such as the femoral artery, at a site remote from the selected portion of the selected region, such as a blood vessel, to be imaged. The catheter 102 may then be advanced through the blood vessels of the patient to the selected imaging site, such as a portion of a selected blood vessel.

Each transducer is composed of piezocomposite structure containing a piezoceramic material and a non-piezoelectric material, such as a polymeric material. The use of this piezocomposite structure can provide improved imaging performance. For instance, piezocomposite structures typically have lower acoustic impedance and higher electric-mechanical coupling coefficient, k₃₃, than piezoceramic materials alone. Because the acoustic impedance is lower, the degree of impedance mismatch between a piezocomposite transducer and the surrounding environment is typically less than transducers employing piezoceramic materials alone. Also, the higher coupling coefficient, k₃₃, allows the piezocomposite transducer to operate over a wider bandwidth of ultrasound energy or to operate with a greater sensitivity to ultrasound energy (or both).

The piezoceramic material of the piezocomposite transducers for use in the embodiments described herein is PIN-PMN-PT (lead indium niobate-lean magnesium niobate-lead titanate) (e.g., Pb(In_{1/2}Nb_{1/2})O₃—Pb(Mg_{1/3}Nb_{2/3})O₃—PbTiO₃.) PIN-PMN-PT typically includes at least 10 wt. % of each component (PIN, PMN, and PT). Any suitable ratios of PIN and PMN to PT can be used so long as the resulting material has suitable piezoelectric properties. Within a crystal (which in some embodiments may be cut to yield multiple transducers), one or both of the ratios of PIN and PMN to PT may vary along the crystal.

The polymeric material used to form the piezocomposite structure can include, for example, an epoxy (for example, Epo-Tek 301-2 available from Epoxy Technology, Bilerica, Mass.), other polymers, and the like.

PIN-PMN-PT has advantages over other piezoceramic materials such as PMN-PT (lead magnesium niobate-lead titanate). Table 1 is a comparison of mean material properties between PIN-PMN-PT and PMN-PT single crystal material as measured over seventeen crystal wafers taken from a crystal boule. It will be recognized that these values are representative, but values for other PIN-PMN-PT and PMN-PT materials may vary.

TABLE 1
Comparison of Mean Properties for 17 PIN-PMN-PT Crystals and
17 PMN-PT Crystals

| | PIN-PMN-PT | |
|---|---|---|
| | crystal | PMN-PT crystal |
| E_{c} (kV/cm) | 5.88 | 2.73 |
| T_{R/T} (° C.) | 110.7 | 94.4 |
| K^{T}₃₃ | 4289 | 5478 |
| d₃₃ (pm/V) | 1301 | 1503 |
| k₃₃ | 0.865 | 0.896 |
| s^{E}₃₃ (×10⁻¹² m²/N) | 41.6 | 50.6 |

The electrical and mechanical coupling coefficient, k₃₃, is one of the most important factors for piezoelectric materials as it directly impacts the bandwidth and sensitivity of a transducer made using this material. Incorporating piezoceramic materials, such as PIN-PMN-PT and PMN-PT) with high k₃₃ facilitates the development of high performance 1-3 composite structures and other useful transducer structures. As seen in Table 1, the coefficient k₃₃ is relatively high for both PIN-PMN-PT and PMN-PT.

One of the key advantages of ternary PIN-PMN-PT crystal over binary PMN-PT is the higher coercivity, E_{C}, which is approximately 6 kV/cm for ternary PIN-PMN-PT crystal; more than double that of binary PMN-PT. In addition, the E_{C} value of ternary crystals is substantially stable for specimens selected throughout the crystal boule. The PIN-PMN-PT crystal has improved linear strain response over a larger field range compared with the binary PMN-PT crystal.

The depoling temperature T_{R/T}, which denotes the upper temperature limit for piezoelectric crystal applications, is increased to 110° C. for PIN-PMN-PT, which is about 20° C. higher than for binary PMN-PT. In fact, the T_{R/T} values for all of the PIN-PMN-PT crystal wafers were greater than 100° C. Thus, PIN-PMN-PT crystals can generally be used at higher temperature than PMN-PT. Moreover, for applications near, or at, room temperature the temperature dependence of performance will typically be substantially reduced for the higher transformation temperature material (i.e., PIN-PMN-PT.)

Table 1 also compares other properties. The values of the relative dielectric constant K₃₃^{T} for PIN-PMN-PT range between 3450 and 5650, from the base to the MPB (morphotropic phase boundary), with an average value of 4290. These K₃₃^{T} values are approximately 20% less than those for binary PMN-PT. The piezoelectric strain coefficient (d₃₃=900-1900 pm/V) is approximately 14% less for the ternary PIN-PMN-PT crystal. There is also a slight decrease (about 3%) in the electromechanical coupling factor (k₃₃=0.83-0.92) for PIN-PMN-PT. Nevertheless, these properties are quite good when compared with conventional piezoceramic materials.

In summary, compared with binary PMN-PT, ternary PIN-PMN-PT crystal has twice the coercive field (E_{C}˜6.0 kV/cm), and has an increase in depoling temperature (T_{R/T}˜100-117° C.) by approximately 20° C. The higher coercive field and depoling temperature can give greater stability in many piezoelectric transducer applications. Other property coefficients, such as dielectric constant, piezoelectric strain coefficient, and coupling factor were slightly reduced for PIN-PMN-PT (as compared to PMN-PT), with increasing stiffness at room temperature. PIN-PMN-PT crystals can be grown in large size and on an industrial scale.

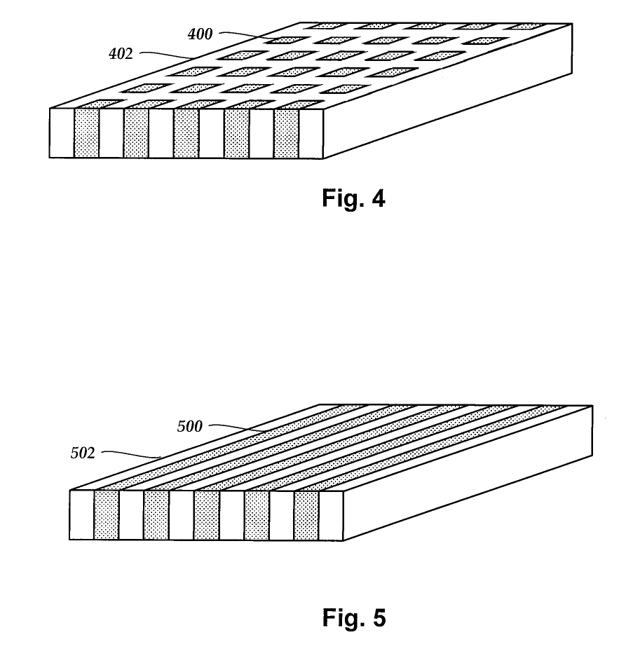

Another factor in transducer design is the frequency of the lateral vibration modes of the transducer structure. Preferably, the lateral vibration modes generated by the kerf structure of a 1-3 composite structure (or other transducer structure) have a frequency that is at least an octave range away from the operating frequency of the transducer. FIG. 4 illustrates a 1-3 composite structure with posts of piezoelectric material (i.e., PIN-PMN-PT) 400 and kerfs containing polymeric material 402 separating the posts. FIG. 5 illustrates a 2-2 composite structure with rows (or columns) of piezoelectric material 500 (i.e., PIN-PMN-PT) and kerfs containing polymeric material 502 separating the rows or columns.

Differing from the bulk piezoceramic material, a1-3 composite structure (or other composite structure) introduces a kerf structure which has a kerf width and is typically filled with a kerf material selected from polymeric materials. The kerf structure often generates undesired lateral vibrations for which the phase is reversed with respect to the primary thickness mode of the transducer vibration. Preferably, the lateral vibration modes generated by the kerf structure of the 1-3 composite structure (or other composite structure including a 2-2 composite structure) have a frequency that is at least an octave range away from the operating frequency. For high frequency transducer, with an operating frequency of at least 40 MHz, the kerf width could be as narrow as less than 4 micrometer.

Conventional composite transducers and arrays operating below 20 MHz are constructed by dicing piezoelectric ceramics and then filling the kerf space with polymer materials to form composite structures. Transducers based on this architecture typically exhibit high bandwidth, high sensitivity, good acoustic impedance matching to tissue, and good array properties (e.g., low inter-element cross talk, low side-lobe levels). However, this “dice-and-fill” method of transducer fabrication generally cannot be used to make array transducers that operate much above about 20 MHz.

The diced feature sizes are difficult to achieve in high frequency transducers. The frequency limitation comes, at least in part, from the frequency of the lateral mode resonance which is determined by the shear wave velocity of the filler material and the width of the dicing cut. The frequency of the first lateral mode in a 1-3 composite structure can be empirically expressed as

$${f}_{l} = \frac{{V}_{T}}{2 {d}_{p}}$$

where f₁ is the frequency of the first lateral mode, V_{T} is the shear wave velocity and dₚ is the kerf width. For example, if the kerf width is 10 μm, which is obtainable by the dicing method, the frequency of the first lateral mode is about 39 MHz, limiting the operating frequency of this composite to about 20 MHz.

Moreover, limited by the thickness of the dicing blade and the dicing process, the “dice-and-fill” process can not fabricate very narrow kerf widths of 4 μm or less for producing high frequency composite transducers with an operating frequency of 40 MHz or more.

Reactive ion etching (RIE) has been used to achieve narrow kerf structures as described, for example, in U.S. Patent Application Publication No. 2007/0038111, incorporated herein by reference. The RIE process, however, typically involves expensive equipment and operation in a clean room. Furthermore, RIE also typically includes a relatively long process time (e.g., up to a several hours) to achieve a kerf depth of, for example, 50 μm or so.

Both dicing and RIE methods can be used to make piezocomposite transducers from PIN-PMN-PT. Instead of dicing or RIE, however, a piezocomposite structure can be formed in a PIN-PMN-PT crystal using laser etching. A kerf width of 4 μm or less can be achieved using laser etching. Previously, attempts to laser etch PMN-PT to form kerfs with widths of 4 μm or less have been relatively unsuccessful due to cracking of the PMN-PT material. Surprisingly, it has been found that kerfs with widths 4 μm or less can be formed in PIN-PMN-PT by laser etching. In at least some laser etching processes, the material of is etched by photochemical ablation.

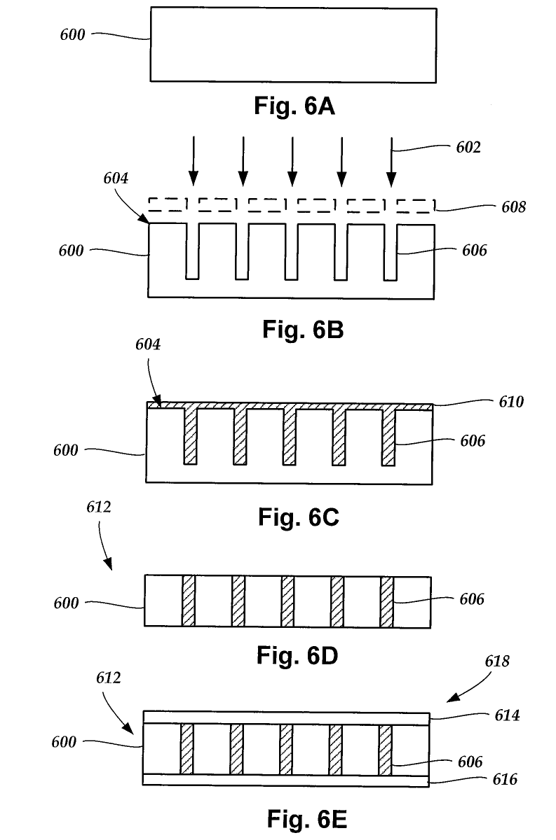

FIGS. 6A-6E illustrate, in cross-section, one embodiment of a method of making a transducer using laser etching. It will be recognized that the dimensions in FIGS. 6A-6E may not be to scale. These Figures are for illustration purposes only.

FIG. 6A illustrates a crystal of PIN-PMN-PT material 600. As one example, a transducer with dimension of approximately 0.53 mm×0.5 mm×0.03 mm can be formed. In some embodiments, multiple transducers can be made from a single crystal (for example, from a single 25 mm×25 mm crystal wafer.) Optionally, the PIN-PMN-PT material 600 is mounted on a substrate (not shown). Any suitable mounting substrate can be used such as silicon, silicon dioxide, and the like and the mounting may be performed using any suitable method include mechanical or adhesive mounting or any combination thereof.

In FIG. 6B, laser light 602 impinges on a surface 604 of the PIN-PMN-PT material 600 to create kerfs 606. The kerf structure determines the type of composite structure. For example, the composite structure may be a 1-3 composite structure or a 2-2 composite structure or some other composite structure (or even a combination of such structures). Each of these composite structures includes an array of piezoelectric elements (e.g., posts, rows, or columns.)

In at least some embodiments, the kerfs do not extend entirely through the PIN-PMN-PT material 600. If the PIN-PMN-PT material is mounted on a substrate, the kerfs may extend entirely through the material.

The kerfs typically have a width of 5 μm or less, 4 μm or less, 3 μm or less, or 2 μm or less. The kerfs have a depth of at least 30 μm, at least 50 μm, or at least 70 μm. The aspect ratio (depth:width) of the crystal pillars is typically greater than 2, or greater than 5, or greater than 10.

In some embodiments, a mask 608 is prepared using known lithographic techniques (or any other suitable mask generation technique). The laser light 602 illuminates the mask that contains the desired kerf pattern and this pattern is imaged onto the PIN-PMN-PT material 600 to create the kerf pattern. Optionally, one or more optical elements (not shown), such as one or more lens, beam expanders, collimators, homogenizers, or any combination thereof, can be used to image the kerf pattern onto the PIN-PMN-PT material.

In other embodiments, a mask is not used and, instead, the laser light 602 directly writes onto the PIN-PMN-PT material 600. For example, the laser, or optics (e.g., a scanning stage) that receives the laser light, scans the laser light 602 over the surface 604 of the PIN-PMN-PT material 600 to generate the kerf pattern. Typically, the scanning process is automated.

Any suitable laser can be used. The resolution of the laser etching procedure is proportional to 2λ, where λ is the laser wavelength. Accordingly, in at least some embodiments, a laser source with a wavelength in range of 150 μm to 300 μm, in the range of 170 μm to 250 μm, or in the range from 193 μm to 248 μm, is used. In at least some embodiments, the laser has an adjustable wavelength. Examples of suitable lasers for the wavelength ranges recited above include excimer lasers and diode-pumped solid state lasers.

In at least some embodiments, the laser is a pulsed laser. Any suitable pulsing rate can be used. In at least some embodiments, the pulsing rate is at least 10 Hz, 50 Hz, 100 Hz, 200 Hz, or 500 Hz.

Any suitable etching rate can be used. In at least some embodiments, the etching rate is 0.05, 0.1, or 0.15 micrometer per laser pulse. By controlling the laser repetition rate, i.e., the pulse rate, the PIN-PMN-PT material can be micromachined to the width and depth for the desired piezocomposite structure. For instance, at a “pulse repeat rate” of 100 Hz and an “etch rate” of 0.1 μm/pulse, the “etching depth rate”=(pulse repeat rate)*(etching rate)=10 μm/sec. At this etching depth rate only about four seconds is needed to make a kerf depth of 40 μm. If the pulse repeat rate=200 Hz, then only 2 seconds is needed. This may be substantially faster than RIE methods.

After forming the kerfs by laser etching, the kerfs 606 are filled using a polymeric material 610, as illustrated in FIG. 6C. Alternatively, another non-piezoelecric material can be used in place of the polymeric material. The polymeric material 610 may be disposed in the kerfs using any suitable method including, but not limited, to spin coating, dip coating, other coating methods, chemical vapor deposition, physical vapor deposition, other deposition methods, and the like.

In some embodiments, the polymeric material 610 may be initially disposed in a solvent for purposes of filling the kerfs 606. The solvent can be removed afterwards leaving the polymeric material 610.

In some embodiments, a monomeric or oligomeric material can be coated or otherwise deposited on the PIN-PMN-PT material and then reacted to form the polymeric material 610. In some of these embodiments, the monomeric or oligomeric material includes two or more different types of materials that may be mixed prior to, or during, coating or deposition and then reacted to create the polymeric material. Examples of such materials include many epoxies with two or more components.

In some embodiments, the polymeric material 610 may be heated to encourage flow into the kerfs 606. The polymeric material 610 can then be cooled to solidify or otherwise fix the material within the kerfs.

In some embodiments, the polymeric material 610 may be cross-linked or otherwise reacted after filling the kerfs using any suitable technique (e.g., heat or ultraviolet activated cross-linking or reacting).

In at least some embodiments, the polymeric material 610 not only fills the kerfs but also extends over at least a portion of the surface 604 as illustrated in FIG. 6C. In other embodiments, the polymeric material may not fill all of the kerfs 606 completely.

After filling the kerfs, the top, or bottom or both, of the PIN-PMN-PT material 600 is optionally lapped to create a piezoelectric composite structure 612, as illustrated in FIG. 6D. If the PIN-PMN-PT is mounted on a substrate, the substrate may be removed.

Electrodes 614, 616 are formed on the piezoelectric composite structure 612 to form the transducer 618, as illustrated in FIG. 6E. In some embodiments, one or both of the electrodes 614, 616, can be patterned to provide an array of transducers.

The PIN-PMN-PT transducers preferably have an operating frequency of at least 20 MHz, at least 30 MHz, at least 40 MHz, at least 50 MHz, or at least 60 MHz.

The piezoelectric transducer can be configured as a single element transducer, an array of transducers, or any other configuration desired. The array of transducers can be, for example, a one-dimension or two-dimensional array. The array can be a linear array, a curved array, a curved linear array, or a phased array.

In some embodiments, the transducer is substantially flattened or planar and configured to transmit or receive ultrasound energy from a planer surface. In other embodiments, the transducer can be shaped or curved in any number of directions and in any manner. The emitting surface of the transducer may be planer, curved, or otherwise shaped.

In addition, various shapes of arrays may be formed. For example, the array may be an annular array of imaging transducers is shown. An alternative annular array includes a central element and annular aperture elements concentrically positioned around the central element. In other embodiments, the array may have rows or columns (or both) with individual transducer elements.

It will be recognized that the transducers described herein can be used in applications other than IVUS or ICE. For example, the transducers may be useful in therapy or treatment of conditions. For example, the transducers may be useful in skin, eye, colon, and other therapies or treatments.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method of making an ultrasound transducer, the method comprising:
providing a piezoelectric crystal of PIN-PMN-PT (lead indium niobate-lead magnesium niobate-lead titanate);
etching a plurality of kerfs into the piezoelectric crystal using a laser, wherein each kerf has a width of no more than 4 μm; and
filling the plurality of kerfs with a non-piezoelectric material to form an array of piezoelectric elements.

2. The method of claim 1, further comprising forming electrodes on the array of piezoelectric elements to form the ultrasound transducer.

3. The method of claim 2, wherein forming electrodes comprises patterning at least one of the electrodes.

4. The method of claim 1, wherein etching a plurality of kerfs comprises pulsing the laser to etch the piezoelectric crystal.

5. The method of claim 4, wherein pulsing the laser comprises removing at least 0.05 μm thickness of piezoelectric material per pulse.

6. The method of claim 1, wherein etching the plurality of kerfs comprises etching the plurality of kerfs to generate a 1-3 composite structure.

7. The method of claim 1, wherein etching the plurality of kerfs comprises etching the plurality of kerfs to generate a 2-2 composite structure.

8. The method of claim 1, wherein etching the plurality of kerfs comprises etching the plurality of kerfs using a mask through which light from the laser is directed.

9. The method of claim 1, wherein etching the plurality of kerfs comprises scanning the laser over the piezoelectric crystal to generate the kerfs.

10. The method of claim 1, wherein the ultrasound transducer has an operating frequency of at least 40 MHz.

11. An ultrasound transducer, comprising:
a piezocomposite structure comprising a plurality of transducer elements separated by kerfs, wherein the transducer elements are PIN-PMN-PT (lead indium niobate-lead magnesium niobate-lead titanate); and
first and second electrodes disposed on the piezocomposite structure.

12. The ultrasound transducer of claim 11, wherein the kerfs have a width of no more than 4 μm.

13. The ultrasound transducer of claim 12, wherein the ultrasound transducer has an operating frequency of at least 40 MHz.

14. The ultrasound transducer of claim 11, wherein the kerfs are formed by laser etching.

15. The ultrasound transducer of claim 11, wherein the piezocomposite structure has a 1-3 composite structure.

16. The ultrasound transducer of claim 11, wherein the piezocomposite structure has a 2-2 composite structure.

17. The ultrasound transducer of claim 11, wherein the ultrasound transducer has a curved emitting surface.

18. The ultrasound transducer of claim 11, wherein the ultrasound transducer comprises an array of transducer elements configured and arranged to independently emit ultrasound pulses.

19. An ultrasound imaging system, comprising:
a catheter, and
the ultrasound transducer of claim 11 disposed in a distal end of the catheter.

20. The ultrasound imaging system of claim 19, further comprising a control module coupled to the catheter and the ultrasound transducer.
